(19) United States
(12) Patent Application Publication
(10) Pub. No.: US 2014/0045740 A1
(43) Pub. Date: Feb. 13, 2014

(54) ANALYSIS OF GLATIRAMER ACETATE

(71) Applicant: Momenta Pharmaceuticals, Inc., Cambridge, MA (US)

(72) Inventors: Joseph Glajch, Nashua, NH (US); Ying Li, Wellesley, MA (US)

(73) Assignee: Momenta Pharmaceuticals, Inc., Cambridge, MA (US)

(21) Appl. No.: 13/964,862

(22) Filed: Aug. 12, 2013

Related U.S. Application Data
(60) Provisional application No. 61/682,443, filed on Aug. 13, 2012.

(51) Int. Cl. G01N 33/68; A61K 38/00

(52) U.S. Cl. CPC G01N 33/6848; A61K 38/00
USPC 514/1.1; 530/300; 436/86

(57) ABSTRACT

The present disclosure relates to methods for assessing molecular weight characteristics of glatiramer acetate using SEC-MALS.

### CROSS-REFERENCE TO RELATED APPLICATIONS

This application claims the benefit of U.S. Provisional Application Ser. No. 61/682,443 filed Aug. 13, 2012, the content of which is incorporated herein by reference in its entirety.

### TECHNICAL FIELD

The present disclosure relates to methods for assessing various molecular weight characteristics of glatiramer acetate.

### BACKGROUND

Glatiramer Acetate is the active ingredient in COPAXONE® (Teva Pharmaceutical Industries Ltd., Israel). According to the COPAXONE® product label, glatiramer acetate (GA) consists of the acetate salts of synthetic polypeptides, containing four naturally occurring amino acids: L-glutamic acid, L-alanine, L-tyrosine, and L-lysine with a reported average molar fraction of 0.141, 0.427, 0.095, and 0.338, respectively. Chemically, GA is designated L-glutamic acid polymer with L-alanine, L-lysine and L-tyrosine, acetate (salt). Its structural formula is:

(Glu,Ala,Lys,Tyr)ₓ.xCH₃COOH(C₅H₉NO₄.C₃H₇NO₂.C₆H₁₄N₂O₂.C₉H₁₁NO₃)ₓ.xC₂H₄O₂ CAS-147245-92-9

According to the COPAXONE® product label, glatiramer acetate has an average molecular weight of 5,000-7,000.

### SUMMARY

The invention is based, at least in part, on the development of an improved approach for assessing certain molecular weight characteristics of glatiramer acetate and methods for assessing whether an amino acid copolymer preparation consisting of glycine, tyrosine, lysine and alanine may be formulated into glatiramer acetate.

The methods described herein entail the use of size exclusion chromatography (SEC) coupled to multi-angle-light scattering and refractive index detectors (SEC-MALS-RI). The methods do not employ column calibration standards.

In the methods described herein SEC-MALS-RI is used to determine the weight-average molecular weight (Mw) using the Rayleigh-Gans-Debye equation with Zimm formalism, described in greater detail below. In this approach the light scattering signal is assumed to proportional to average molecular weight and sample concentration at any point in a chromatogram, and specific increment of refractive index (dn/dc). Thus, light scattering detectors coupled with a refractive index detector as a concentration detector can accurately determine the average molecular weight for any point in the chromatogram and analysis of the entire chromatographic distribution can be used to determine the weight—average molecular weight (Mw) when an appropriate value for dn/dc is obtained. Mp, the average molecular weight at the apex of the sample peak, can also be determined as can the number-average molecular weight, Mn, and the Z-average molecular weight, Mz.

Because the methods described herein can be used to assess Mp, Mn, Mz and Ip, they can provide a detailed profile of a sample of GA. This detailed profile can be used for comparing a composition comprising GA to a reference value or range. For example the determined value for one or more of the parameters (e.g., one or more of Mp, Mn, Mz and Ip) can be compared to reference value or range for the one or more parameters. The reference value or range can be a specification for commercial release of GA or for processing a composition comprising GA to a GA drug substance or GA drug product.

The determined value for one or more of the parameters of a sample of a batch of GA can also be compared to a reference value or range for one or more of the parameters in order to determine if certain additional steps should take place. For example, if the determined value of the one or more parameters has a preselected relationship with the reference value or range for the parameter(s) the methods described herein can include: classifying, selecting, accepting, discarding, releasing, or withholding a batch of glatiramer acetate; reprocessing a batch of glatiramer acetate through a previous manufacturing step; processing a batch of glatiramer acetate into drug product, shipping the product from a batch of glatiramer acetate, moving the batch of glatiramer acetate to a new location; or formulating, labeling, packaging, selling, offering for sell, or releasing a batch of glatiramer acetate into commerce.

Because the methods described herein can be used to assess Mp, Mn, Mz and Ip, they can provide a detailed profile of a sample of a composition comprising a copolymer (e.g., a copolymer having a molar ratio of L-glutamic acid:L-alanine:L-tyrosine:L-lysine of 0.141:0.427:0.095:0.338). This detailed profile can be used for comparing a composition comprising such a copolymer to a reference value or range. For example, the determined value for one or more of the parameters (e.g., one or more of Mp, Mn, Mz and Ip) can be compared to reference value or range for the one or more parameters. The reference value or range can be one for determining whether the copolymer is GA.

In some cases a sample of copolymer can have an Mp of 5000-9000 Daltons (e.g., 6000-8000, 6500-7500, 7000-7500) and one or more of: an Mn of 5500-8600 Daltons, an Mw of 7500-11800 Daltons (e.g., 8000-11000, 8500-10500), an Mz of 10400-16400 Daltons (11000-16000, 12000-15000), and an Ip of 1.28-1.52 (1.30-1.40).

The determined value for one or more of the parameters of a sample of a batch of a copolymer can also be compared to a reference value or range for one or more of the parameters in order to determine if certain additional steps should take place. For example, if the determined value of the one or more parameters has a preselected relationship with the reference value or range for the parameter(s) the methods described herein can include: classifying, selecting, accepting, discarding, releasing, or withholding the batch of copolymer; reprocessing a batch of copolymer through a previous manufacturing step; processing a batch of copolymer to glatiramer acetate into drug product, shipping the product from a batch of copolymer, moving the batch of copolymer to a new location; or formulating, labeling, packaging, selling, offering for sell, or releasing a batch of copolymer into commerce.

Described herein is a method of preparing a glatiramer acetate drug product, the method comprising: obtaining a sample of a batch of glatiramer acetate; using SEC-MALS (e.g., SEC-MAL-RI) to determine the average molar mass at the peak (Mp) of the glatiramer acetate in the sample and to determine one or more of: the weight-average molar mass (Mw) number-average molar mass (Mn), the Z-average molar mass (Mz) and the polydispersity (Mw/Mn) of the glatiramer acetate in the sample; and processing at least a portion of the batch to prepare a glatiramer acetate drug product if the determined Mp is between 5000 and 9000 Da. In some cases the Mw, Mn, Mz, and Mp are calculated using a dn/dc value of 0.197 mol/gm.

In some cases the method further comprises: processing at least a portion of the batch to prepare glatiramer acetate drug product if the Mp is between 5000 and 9000 and one or more (two or more, three or more, or all four) of the following criteria are met:
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

In some cases: the processing step comprising adding a composition comprising mannitol to the at least a portion of the batch; processing at least a portion of the batch to prepare a glatiramer acetate drug product if the determined Mw is between 7500 and 11800 Dalton (e.g., 8000-11000, 8500-10500); processing at least a portion of the batch to prepare a glatiramer acetate drug product if the determined Mn is between 5500 and 8600 (e.g., 6000-8000, 6500-8000); processing at least a portion of the batch to prepare a glatiramer acetate drug product if the determined Mz is between 10400 and 16400 Daltons (e.g., 11000-16000, 12000-15000); and processing at least a portion of the batch to prepare a glatiramer acetate drug product if the determined polydispersity is between 1.28-1.52 (e.g., 1.28 and 1.44, 1.30-1.40)

In some cases the batch of glatiramer acetate is prepared by a method comprising: polymerizing N-carboxy anhydrides of L-alanine, benzyl-protected L-glutamic acid, trifluoroacetic acid (TFA)-protected L-lysine, and L-tyrosine to generate a protected copolymer (Intermediate-1); treating the protected copolymer to partially depolymerize the protected copolymer and to deprotect benzyl protected groups thereby producing Intermediate-2; treating Intermediate-2 to deprotect TFA-protected lysines thereby producing Intermediate-3; and processing Intermediate-3 to produce glatiramer acetate.

Also described is a method of identifying a batch of a composition comprising an amino acid copolymer of L-glutamic acid, L-alanine, L-tyrosine and L-lysine as comprising glatiramer acetate, the method comprising: obtaining a sample of a batch of the amino acid copolymer; using SEC-MALS-RI to determine the average molar mass at the peak (Mp) of the copolymer and to determine one or more of: the weight-average molar mass (Mw) number-average molar mass (Mn), the Z-average molar mass (Mz) and the polydispersity (Mw/Mn) of the glatiramer acetate in the sample; and identifying the batch of copolymer as comprising glatiramer acetate if the determined Mp is between 5000 and 9000. In some cases the Mw, Mn, Mz, and Mp are calculated using a dn/dc value of 0.197 mol/gm.

In some cases the method further includes identifying the batch as comprising glatiramer acetate if the Mp is between 5000 and 9000 and one or more (e.g., two or more, three or more, or all four) of the following criteria are met:
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

In various embodiments the method comprises: identifying the batch as comprising glatiramer acetate if the determined Mw is between 7500 and 11800; identifying the batch as comprising glatiramer acetate if the determined Mn is between 5500 and 8600; identifying the batch as comprising glatiramer acetate if the determined Mz is between 10400 and 16400; and identifying the batch as comprising glatiramer acetate if the determined polydispersity is between 1.28 and 1.44.

In some cases: the batch of copolymer is prepared by a method comprising: polymerizing N-carboxy anhydrides of L-alanine, benzyl-protected L-glutamic acid, trifluoroacetic acid (TFA)-protected L-lysine, and L-tyrosine to generate a protected copolymer (Intermediate-1); treating the protected copolymer to partially depolymerize the protected copolymer and to deprotect benzyl protected groups thereby producing Intermediate-2; treating Intermediate-2 to deprotect TFA-protected lysines thereby producing Intermediate-3; and processing Intermediate-3 to produce a copolymer.

Also described is a method for preparing a pharmaceutical composition comprising glatiramer acetate, comprising: polymerizing N-carboxy anhydrides of L-alanine, benzyl-protected L-glutamic acid, trifluoroacetic acid (TFA)-protected L-lysine, and L-tyrosine to generate a protected copolymer (Intermediate-1); treating the protected copolymer to partially depolymerize the protected copolymer and to deprotect benzyl protected groups thereby producing Intermediate-2; treating Intermediate-2 to deprotect TFA-protected lysines thereby producing Intermediate-3; and processing Intermediate-3 to produce a batch of glatiramer acetate, wherein the improvement comprises: obtaining a sample of the batch of glatiramer acetate; using SEC-MALS-RI to determine the molar mass (Mp) at the peak of the glatiramer acetate in the sample and to determine one or more of: the weight-average molar mass (Mw) number-average molar mass (Mn), the Z-average molar mass (Mz) and the polydispersity (Mw/Mn) of the glatiramer acetate in the sample; and processing at least a portion of the batch of GA to prepare a pharmaceutical composition comprising glatiramer acetate if the determined Mp is between 5000 and 9000. In some cases the Mw, Mn, Mz, and Mp are calculated using a dn/dc value of 0.197 mol/gm.

In some cases the method further comprises: processing at least a portion of the batch to prepare a pharmaceutical composition if the Mp is between 5000 and 9000 and one or more (two or more, three or more, or all four) of the following criteria are met:
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

In some cases: the processing step comprising adding a composition comprising mannitol to the at least a portion of the batch; processing at least a portion of the batch to prepare a a pharmaceutical composition if the determined Mw is between 7500 and 11800 Dalton (e.g., 8000-11000, 8500-10500); processing at least a portion of the batch to prepare a pharmaceutical composition if the determined Mn is between 5500 and 8600 (e.g., 6000-8000, 6500-8000); processing at least a portion of the batch to prepare a pharmaceutical composition if the determined Mz is between 10400 and 16400 Daltons (e.g., 11000-16000, 12000-15000); and processing at least a portion of the batch to prepare a pharmaceutical composition if the determined polydispersity is between 1.28-1.52 (e.g., 1.28 and 1.44, 1.30-1.40).

In some cases a sample of copolymer can have an Mp of 5000-9000 Daltons (e.g., 6000-8000, 6500-7500, 7000-7500) and one or more of: an Mn of 5500-8600 Daltons, an Mw of 7500-11800 Dlatons (e.g., 8000-11000, 8500-10500), an Mz of 10400-16400 Daltons (11000-16000, 12000-15000), and an Ip of 1.28-1.52 (1.30-1.40).

In some cases the methods described herein can be used to determine whether a copolymer, e.g., a copolymer of L-glutamic acid, L-alanine, L-tyrosine and L-lysine, has characteristics consistent with GA. Copolymers that are not consistent with GA include ones which do not have, e.g., the same ratio of L-glutamic acid, L-alanine, L-tyrosine, and L-lysine as GA, and/or are otherwise distinguishable from GA, for example, by way of molecular weight, molecular weight distribution, pyro-glutamate content, the presence of other material (e.g., agents not present in GA or that are present in GA but at controlled or regulated levels) etc., and thus, are distinguishable from GA. In some cases copolymers that are not consistent with GA have one or more of the following characteristics: a Mn outside of 5500-8600 Da; a Mw outside 7500-11800 Da; a Mz outside 10400-16400 Da; an Ip outside 1.28-1.44; a peak molecular weight outside of 5,000-9,000 Da; a pyro-glutamate content outside of 2,000-7,000 ppm; a pyro-glutamate content outside of 2,500-5,500 ppm; a pyro-glutamate content outside of 3,000-5,000 ppm; a pyro-glutamate content outside of 3,500-4,500 ppm; a pyro-glutamate content outside of 5,000-9,000 ppm; a pyro-glutamate content outside of 6,500-7,500 ppm; and molar ratio of L-glutamic acid:L-alanine:L-tyrosine:L-lysine outside of: 0.141:0.427:0.095:0.338.

As used herein, a “copolymer”, “amino acid copolymer” or “amino acid copolymer preparation” is a heterogeneous mixture of polypeptides comprising a defined plurality of different amino acids (typically between 2-10, e.g., between 3-6, different amino acids). A copolymer may be prepared from the polymerization of individual amino acids. The term “amino acid” is not limited to naturally occurring amino acids, but can include amino acid derivatives and/or amino acid analogs. For example, in an amino acid copolymer comprising tyrosine amino acids, one or more of the amino acids can be a homotyrosine. Further, an amino acid copolymer having one or more non-peptide or peptidomimetic bonds between two adjacent residues is included within this definition. A copolymer is non-uniform with respect to the molecular weight of each species of polypeptide within the mixture.

A glatiramer acetate drug product can comprises glatiramer acetate and mannitol.

As used herein, a “reference value” is a range or level of at least one molecular weight parameter (e.g., Mw, Mz, Mn, Mp or Ip). In some instances, a reference value is a specification for commercial release of a drug product comprising GA. For example, the specification for commercial release can be the specification required by the U.S. Food & Drug Administration (FDA), the European Medicines Agency (EMA), or the U.S. Pharmacopeial Convention (USP), e.g., for the pharmaceutical release of GA, or as provided on the label for GA, e.g., as approved by the FDA, the EMA, or the USP.

As used herein, the term determine, e.g., in the molecular weight analysis described herein, includes, for example, monitor, assaying, measure, assess, analyze, calculate, detect, review, evaluate, correlate and/or estimate.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE FIGURES

FIG. 1 is a graph showing the light scattering profile (LS), the refractive index profile (RI) and the calculated molar mass for a sample of COPAXONE® analyzed by SEC-MALS-RI using an embodiment of the method described herein.

### DETAILED DESCRIPTION OF THE INVENTION

The present invention provides methods for determining certain molecular weight characteristics of glatiramer acetate and methods for determining whether a glatiramer acetate preparation or a sample, lot, or batch thereof, conforms to a reference value for glatiramer acetate. Thus, methods described herein can be used to distinguish glatiramer acetate from certain non-conforming copolymers.

### Methods for Manufacture of Glatiramer Acetate

Generally, the process for the manufacture of glatiramer acetate includes three steps:
Step (1): polymerization of N-carboxy anhydrides of L-alanine, benzyl-protected L-glutamic acid, trifluoroacetic acid (TFA) protected L-lysine and L-tyrosine (collectively referred to as NCAs) to result in a protected copolymer,
Step (2): depolymerization and benzyl deprotection of the protected copolymer using hydrobromic acid in acetic acid, and
Step (3): deprotection of the TFA-protected lysines on the product copolymers followed by purification and drying of the isolated drug substance.

In Step (1) of the manufacturing method, the NCAs are co-polymerized in a predetermined ratio using diethylamine as an initiator. Upon consumption of the NCA components, the reaction mixture is quenched in water. The resulting protected polymer (Intermediate-1) is isolated and dried. In Step (2), the protected polymer (Intermediate-1) is treated with anhydrous 33% HBr in acetic acid (HBr/AcOH). This results in the cleavage of the benzyl protecting group on the glutamic acid as well as cleavage of peptide bonds throughout the polymer, resulting in a partially depolymerized product (Intermediate-2) with a reduced molecular weight relative to the parent Intermediate-1 polymer. After the reaction is quenched with cold water, the product polymer is isolated by filtration and washed with water. The Intermediate-2 material is dried before proceeding to Step (3). In Step (3), Intermediate-2 is treated with aqueous piperidine to remove the trifluoroacetyl group on the lysine. The resulting copolymer (Intermediate-3) is subsequently purified using diafiltration/ultrafiltration and the resulting acetate salt dried to produce Glatiramer Acetate drug substance.

Methods for the manufacture of glatiramer acetate have been described in the following publications: U.S. Pat. No. 3,849,550; WO 95/031990 and US 2007-0021324.

Other than molecular weight and amino acid composition, which are specified on the approved label for the product, the label and other available literature for COPAXONE®, referred to herein as the reference drug lot (RLD), do not provide detailed information about the physiochemical characteristics of the product. Various patent applications, including US 2010/0210817, describe methods for assessing the molecular weight of GA using SEC and molecular weight standards.

Because glatiramer acetate is a complex mixture of peptides of varying size and sequence it is challenging to assess the molecular weight distribution. One approach is to use a series of polypeptide molecular weight standards in conjunction with SEC-RI or SEC-UV. However, the molecular weight range of GA is very broad, and it can be difficult to arrive at a set of polypeptide molecular weight standards that are sufficiently representative of the molecular weight range of GA. Moreover, since each polypeptide molecular weight standard is a single molecular entity, they cannot adequately represent the complexity of GA for each slice with same hydrodynamic volume.

SEC-MALS-RI can be used to determine the weight-average molecular weight (Mw) using the Rayleigh-Gans-Debye equation with Zimm formalism using Equation (I) in which light scattering signal in proportional to average molecular weight and sample concentration at any point in the chromatogram and specific increment of refractive index (dn/dc). Thus, light scattering detectors coupled with a refractive index detector used as a concentration detector can accurately determine the average molecular weight at any point in the distribution if dn/dc is known.

R(θ)=K*MCP(θ)[1-2A₂MCP(θ)] (I)

### Wherein:

R(θ) is excess (from the solute alone) Rayleigh ratio. The ratio of the scatter and incident light intensity, corrected for size of scattering volume and distance from scattering volume.
M is molar mass (molecular weight).
C is analyte concentration.
K* is the Rayleigh ratio constant, determined according to Equation (II).

K*=(4π²(nₒ)²/N_{A}(λₒ)⁴)(dn/dc) (II)

nₒ is the solvent refractive index.
N_{A} is Avogadro's number.
I₀ is the vacuum wavelength of incident light.
dn/dc is the specific refractive index increment.
P(θ) is the form factor or scattering function and relates the angular variation in scattering intensity to the mean square radius (r_{g}) of the particle.
A₂ is a second viral coefficient, a measure of solute solvent interaction.

From this analysis, number average molecular weight (Mn), z average molecular weight (Mz), polydispersity (Mw/Mn), and peak molecular weight (Mp) can be determined.

The label information for COPAXONE® states that the average molecular weight is 5000-9000, but does not explain whether this means that average molecular weight at the peak (Mp) falls between 5000 and 9000, that weight-average molecular weight (Mw) falls in this range, that the number-average molecular weight (Mn) falls in this range or some other assessment of “average” molecular weight falls within this range.

Described below is the development of a SEC-MALS-RI technique that can provide a fuller assessment of the molecular weight characteristic of GA.

### Example 1

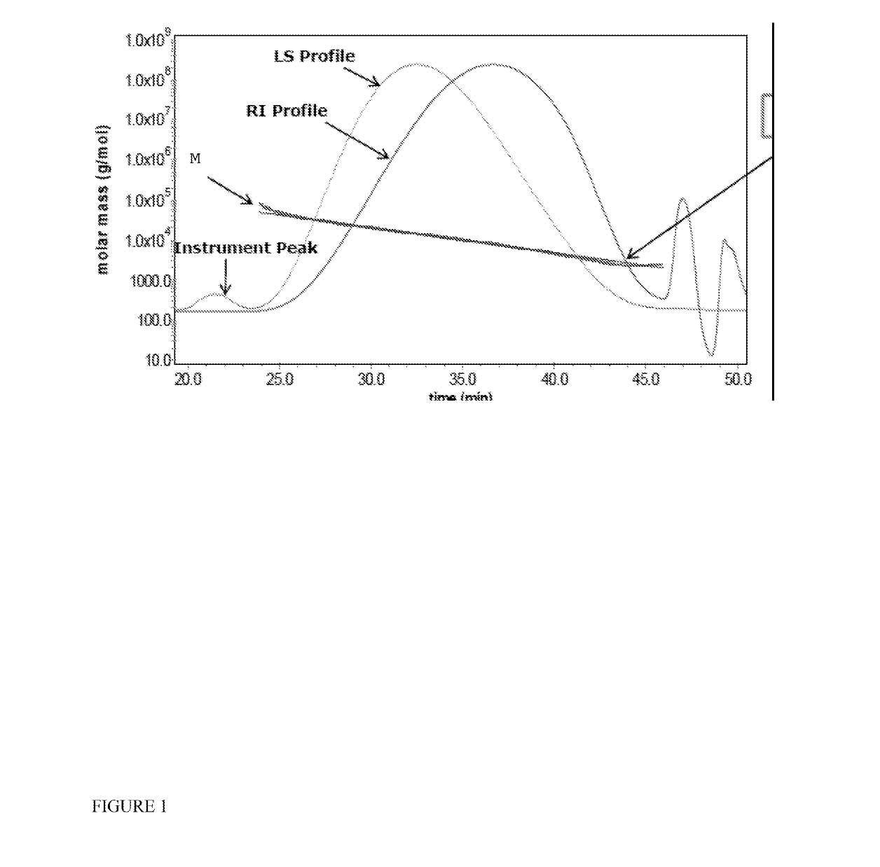

A wide range of SEC columns and mobile phases were investigated in order arrive at a system that was robust, reproducible and provided a sufficient spread of the various species in GA. This investigation resulted in the selection of Tosoh TSKgel G3000swxl and G2000swxl columns working in tandem using the mobile phase of 0.1M sodium phosphate and 0.3M sodium chloride at pH3.5. FIG. 1 is trace showing the light scattering profile (LS), the refractive index profile (RI) and the calculated molar mass (M) for a sample of COPAXONE® analyzed by SEC-MALS-RI using the selected system.

### FIG. 1

The specific refractive index increment (dn/dc) is needed for the accurate analysis of molecular weight using the SEC-MALS-RI technique. The dn/dc was measured in the mobile phase of 0.1M sodium phosphate and 0.15M sodium chloride at pH 3.5. Two lots of Glatiramer Acetate were used to measure dn/dc. Each lot of sample solution was prepared in duplicate and measured independently on two RI detectors. The average number of dn/dc was found to be 0.197 mL/g with and RSD of 1.54%. The dn/dc for most peptides and proteins is assumed to be 0.185 mL/g, a value that is significantly different from that found for GA. A number of factors, including electronegativity and molecular structure can impact the dn/dc value. The unusual amino acid content of GA might account for higher observed dn/dc.

If the standard dn/dc value of 0.185 mL/g was used instead of 0.197 mL/g, the values for Mn, Mw, Mz and Mp would be incorrect. In some cases the calculated Mp was outside or barely within the label range for samples of Copaxone® under certain conditions.

### Example 2

The SEC-MALS-RI method described above was used to analyze samples from 17 different lots of COPAXONE®. In this analysis a value of 0.197 mL/g was used for dn/dc. The results of this analysis are summarized in Table 1.

TABLE 1
N = 17

| | Mn | Mw | Mz | Ip | Mp |
|---|---|---|---|---|---|
| Minimum | 6463 | 8874 | 12220 | 1.327 | 6154 |
| Maximum | 7478 | 10270 | 14260 | 1.385 | 7433 |

The data provided in Table 1 can be used to assess a batch of a copolymer to determine if it conforms to GA. Thus, a sample that conforms to GA can have and Mp of 5000-9000 and one or more of: an Mn of 5500-8600, an Mw of 7500-11800, an Mz of 10400-16400, and an Ip of 1.28-1.52.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of preparing a glatiramer acetate drug product, the method comprising:
obtaining a sample of a batch of glatiramer acetate;
using SEC-MALS-RI to determine the average molar mass at the peak (Mp) of the glatiramer acetate in the sample and to determine one or more of: the weight-average molar mass (Mw) number-average molar mass (Mn), the Z-average molar mass (Mz) and the polydispersity (Mw/Mn) of the glatiramer acetate in the sample; and
processing at least a portion of the batch to prepare a glatiramer acetate drug product if the determined Mp is between 5000 and 9000 Da.

2. The method of claim 1 wherein the Mw, Mn, Mz, and Mp are calculated using a dn/dc value of 0.197 mol/gm.

3. The method of claim 1 further comprising:
processing at least a portion of the batch to prepare glatiramer acetate drug product if the Mp is between 5000 and 9000 and one or more of the following criteria are met:
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

4. The method of claim 3 comprising processing at least a portion of the batch to prepare glatiramer acetate drug product if two of the following criteria are met:
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

5. The method of claim 4 comprising processing at least a portion of the batch to prepare glatiramer acetate drug product if three of following criteria are met.
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

6. The method of claim 3 where in the processing step comprising adding a composition comprising mannitol to the at least a portion of the batch.

7. The method of claim 3 comprising processing at least a portion of the batch to prepare a glatiramer acetate drug product if the determined Mw is between 7500 and 11800.

8. The method of claim 3 comprising processing at least a portion of the batch to prepare a glatiramer acetate drug product if the determined Mn is between 5500 and 8600.

9. The method of claim 3 comprising processing at least a portion of the batch to prepare a glatiramer acetate drug product if the determined Mz is between 10400 and 16400.

10. The method of claim 3 comprising processing at least a portion of the batch to prepare a glatiramer acetate drug product if the determined polydispersity is between 1.28 and 1.44.

11. The method of claim 1, wherein the batch of glatiramer acetate is prepared by a method comprising:
polymerizing N-carboxy anhydrides of L-alanine, benzyl-protected L-glutamic acid, trifluoroacetic acid (TFA)-protected L-lysine, and L-tyrosine to generate a protected copolymer (Intermediate-1);
treating the protected copolymer to partially depolymerize the protected copolymer and to deprotect benzyl protected groups thereby producing Intermediate-2;
treating Intermediate-2 to deprotect TFA-protected lysines thereby producing Intermediate-3; and
processing Intermediate-3 to produce glatiramer acetate.

12. A method of identifying a batch of a composition comprising an amino acid copolymer of L-glutamic acid, L-alanine, L-tyrosine and L-lysine as comprising glatiramer acetate, the method comprising:
obtaining a sample of a batch of the amino acid copolymer;
using SEC-MALS-RI to determine the average molar mass at the peak (Mp) of the copolymer and to determine one or more of: the weight-average molar mass (Mw) number-average molar mass (Mn), the Z-average molar mass (Mz) and the polydispersity (Mw/Mn) of the glatiramer acetate in the sample; and
identifying the batch of copolymer as comprising glatiramer acetate if the determined Mp is between 5000 and 9000.

13. The method of claim 12 wherein the Mw, Mn, Mz, and Mp are calculated using a dn/dc value of 0.197 mol/gm.

14. The method of claim 12 further comprising:
identifying the batch as comprising glatiramer acetate if the Mp is between 5000 and 9000 and one or more of the following criteria are met:
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

15. The method of claim 14 comprising identifying the batch as comprising glatiramer acetate if two of the following criteria are met:
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

16. The method of claim 14 comprising identifying the batch as comprising glatiramer acetate if three of the following criteria are met.
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

17. The method of claim 12 comprising identifying the batch as comprising glatiramer acetate if the determined Mw is between 7500 and 11800

18. The method of claim 12 comprising identifying the batch as comprising glatiramer acetate if the determined Mn is between 5500 and 8600.

19. The method of claim 12 comprising identifying the batch as comprising glatiramer acetate if the determined Mz is between 10400 and 16400.

20. The method of claim 12 comprising identifying the batch as comprising glatiramer acetate if the determined polydispersity is between 1.28 and 1.44.

21. The method of claim 12, wherein the batch of copolymer is prepared by a method comprising:
polymerizing N-carboxy anhydrides of L-alanine, benzyl-protected L-glutamic acid, trifluoroacetic acid (TFA)-protected L-lysine, and L-tyrosine to generate a protected copolymer (Intermediate-1);
treating the protected copolymer to partially depolymerize the protected copolymer and to deprotect benzyl protected groups thereby producing Intermediate-2;
treating Intermediate-2 to deprotect TFA-protected lysines thereby producing Intermediate-3; and
processing Intermediate-3 to produce a copolymer.

22. A method for preparing a pharmaceutical composition comprising glatiramer acetate (GA), comprising: polymerizing N-carboxy anhydrides of L-alanine, benzyl-protected L-glutamic acid, trifluoroacetic acid (TFA)-protected L-lysine, and L-tyrosine to generate a protected copolymer (Intermediate-1); treating the protected copolymer to partially depolymerize the protected copolymer and to deprotect benzyl protected groups thereby producing Intermediate-2; treating Intermediate-2 to deprotect TFA-protected lysines thereby producing Intermediate-3; and processing Intermediate-3 to produce a batch of GA,
wherein the improvement comprises:
obtaining a sample of the batch of GA;
using SEC-MALS-RI to determine the molar mass (Mp) at the peak of the GA in the sample and to determine one or more of: the weight-average molar mass (Mw) number-average molar mass (Mn), the Z-average molar mass (Mz) and the polydispersity (Mw/Mn) of the GA in the sample; and
processing at least a portion of the batch of GA to prepare a pharmaceutical composition comprising glatiramer acetate if the determined Mp is between 5000 and 9000.

23. The method of claim 22 wherein the Mw, Mn, Mz, and Mp are calculated using a dn/dc value of 0.197 mol/gm.

24. The method of claim 22 further comprising:
processing at least a portion of the batch to prepare a pharmaceutical composition comprising glatiramer acetate if the Mp is between 5000 and 9000 and one or more of the following criteria are met:
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

25. The method of claim 24 comprising processing at least a portion of the batch to prepare a pharmaceutical composition comprising glatiramer acetate if two of the following criteria are met:
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

26. The method of claim 24 comprising processing at least a portion of the batch to prepare a pharmaceutical composition comprising glatiramer acetate if three of following criteria are met.
(a) the determined Mw is between 7500 and 11800;
(b) the determined Mn is between 5500 and 8600;
(c) the determined Mz is between 10400 and 16400; and
(d) the determined polydispersity is between 1.28 and 1.44.

27. The method of claim 24 where in the processing step comprises adding a composition comprising mannitol to the at least a portion of the batch.

28. The method of claim 24 comprising processing at least a portion of the batch to prepare a pharmaceutical composition comprising glatiramer acetate if the determined Mw is between 7500 and 11800

29. The method of claim 24 comprising processing at least a portion of the batch to prepare a pharmaceutical composition comprising glatiramer acetate if the determined Mn is between 5500 and 8600.

30. The method of claim 24 comprising processing at least a portion of the batch to prepare a pharmaceutical composition comprising glatiramer acetate if the determined Mz is between 10400 and 16400.

31. The method of claim 24 comprising processing at least a portion of the batch to prepare a pharmaceutical composition comprising glatiramer acetate if the determined polydispersity is between 1.28 and 1.44.
